(19) United States
(12) Patent Application Publication
(10) Pub. No.: US 2018/0177991 A1
(43) Pub. Date: Jun. 28, 2018

(54) MICRONEEDLE APPLICATOR

(71) Applicant: INDUSTRY-ACADEMIC COOPERATION FOUNDATION, YONSEI UNIVERSITY, Seoul (KR)

(72) Inventors: Hyung Il JUNG, Seoul (KR); Hui Suk YANG, Seoul (KR)

(73) Assignee: INDUSTRY-ACADEMIC COOPERATION FOUNDATION, YONSEI UNIVERSITY, Seoul (KR)

(21) Appl. No.: 15/852,849

(22) Filed: Dec. 22, 2017

(30) Foreign Application Priority Data
Dec. 26, 2016 (KR) 10-2016-0179405

(51) Int. Cl. A61M 37/00

(52) U.S. Cl. CPC A61M 37/0015; A61M 2037/0023

(57) ABSTRACT

A microneedle applicator for infiltrating microneedles into skin and delivering a drug into the skin is disclosed. The microneedle applicator includes an application portion configured to press the microneedles and has, at one side thereof, a curved surface extending in a direction of applying the microneedles to the skin, a coupler having the microneedles disposed toward the skin at one surface thereof and having the other surface thereof at the curved surface, and a presser extending toward the other side of the application portion and configured to provide a pressing force to the application portion. The application portion is rolled on the skin so that the microneedles are sequentially applied to the skin in a longitudinal direction of the curved surface, and the microneedles are detached from the coupler and applied to the skin.

### CROSS-REFERENCE TO RELATED APPLICATION

This application claims priority to and the benefit of Korean Patent Application No. 2016-0179405, filed on Dec. 26, 2016, the disclosure of which is incorporated herein by reference in its entirety.

### BACKGROUND

### 1. Field of the Disclosure

The present disclosure relates to a microneedle applicator for attaching a microneedle to skin.

### 2. Discussion of Related Art

Numerous drugs and therapeutic agents for treating diseases have been developed. However, in delivering drugs into the body, a problem of passing through a biological barrier (for example, skin, oral mucosa, blood-brain barrier, and the like) and a problem of drug delivery efficiency still remain as points to be improved.

Generally, drugs are orally administered in the form of a tablet or capsule. However, drugs cannot be effectively delivered only by such administration methods because numerous drugs are digested or absorbed in a gastrointestinal tract or lost due to a metabolic process in liver. Further, some drugs cannot be effectively diffused through intestinal mucosa. Patient compliance is also a problem (for example, in a case of a critical patient who has to take medications at a predetermined interval or is unable to take medications).

Another known method for drug delivery is using a conventional needle. While this method is more effective in comparison to oral administration, the method has problems of causing pain at a site of injection, local damage to skin, bleeding, infection at a site of injection, and the like.

To solve the above problems, various microstructures including a microneedle have been developed. The microneedles developed so far have mostly been used for in vivo drug delivery, blood collection, in vivo analyte detection, and the like.

However, conventionally, a microneedle is manufactured in the form of a patch for the microneedle to be more accurately infiltrated into skin, and the microneedle in the form of a patch is forcibly attached to skin in a vertical direction. In such a conventional case, microneedles arranged in an array form are applied to a predetermined area of skin at once.

In such a method, due to application of a vertical force, microneedle patches cannot be stacked, and thus there is a problem in that an application portion needs to be charged with the patches each time the patches are being applied.

Also, a strong force has to be applied for entire microneedles arranged in an array form to be delivered by a single force and be equally infiltrated into skin, and this, as a result, causes pain.

Additionally, in such a conventional case, because microneedles in the form of a patch are applied to a predetermined area by a single force, there are problems in that a limitation exists in terms of an application area and it is difficult for the microneedles to be accurately applied to bent or non-uniform portions of skin such as wrinkles and joint regions.

### SUMMARY OF THE DISCLOSURE

It is an aspect of the present disclosure to provide a microneedle applicator capable of accurately applying a microneedle to an uneven surface of skin.

It is another aspect of the present disclosure to provide a microneedle applicator capable of infiltrating a microneedle into skin by a small force without causing pain.

It is still another aspect of the present disclosure to provide a microneedle applicator capable of infiltrating only microneedles of the microneedle provided in the form of a patch.

According to an aspect of the present disclosure, there is provided a microneedle applicator for infiltrating microneedles into skin and delivering drug into the skin, the microneedle applicator including an application portion configured to press the microneedles and having, at one side thereof, a curved surface extending in a direction in which the microneedles are applied to the skin, a coupler having the microneedles disposed toward the skin at one surface thereof and having the other surface thereof at the curved surface, and a presser extending toward the other side of the application portion and configured to provide a pressing force to the application portion, wherein the application portion is rolled on the skin so that the microneedles are sequentially applied to the skin in a longitudinal direction of the curved surface, and when the microneedles are applied to the skin in the longitudinal direction of the curved surface, the microneedles are detached from the coupler and applied to the skin.

The coupler may be formed of an adhesive layer, the adhesive layer may be formed such that an adhesive force at the one surface at which the microneedles are formed is weaker than an adhesive force at the other surface which is disposed at the curved surface, and when the microneedles are applied to the skin in the longitudinal direction of the curved surface, the microneedles may be detached from the adhesive layer.

The microneedle applicator may further include a supporter having one surface at which the microneedles are supported and the other surface detachably coupled to the coupler, wherein, when the microneedles are applied to the skin in the longitudinal direction of the curved surface, the supporter may be detached from the coupler so that the microneedles are applied to the skin.

The coupler may include a coupling protrusion protruding outward from the curved surface in a radial direction, and a protrusion accommodating groove corresponding to the coupling protrusion may be formed at the other surface of the supporter coupled to the coupler for the coupling protrusion to be inserted thereinto and the coupler and the supporter to be coupled.

The coupling protrusion may protrude in the same direction as a direction in which the application portion rolls, and when the application portion moves toward any one side in the longitudinal direction of the curved surface, the microneedles may be applied to the skin, and the supporter may remain coupled to the coupler.

The application portion may allow the microneedles disposed between the coupler and the skin to come into linear contact with the skin and apply the microneedles to the skin, and the application portion may roll on the skin so that a line at which the skin and the microneedles come into contact moves along the curved surface of the application portion.

The microneedle applicator may further include a connector configured to detachably connect the application portion and the presser, and by being rolled on the skin, the application portion may sequentially press the microneedles in a direction in which the application portion rotates and sequentially apply the microneedles to the skin.

A longitudinal cross-section of the curved surface may have any one of an arc shape, an elliptical shape, and a parabolic shape.

The microneedle applicator may include a supporter coupled to one surface of an adhesive portion having the microneedles formed at the other surface, include protrusions protruding to a predetermined height from the supporter or the adhesive portion at positions in one-to-one correspondence with the microneedles so that the microneedles are disposed thereat, and by being rolled on the skin, the application portion may sequentially press the microneedles corresponding to the protrusions in a direction in which the application portion rotates and sequentially apply the microneedles to the skin.

The application portion may include protrusions protruding to a predetermined height from the curved surface at positions in one-to-one correspondence with the microneedles so that the microneedles are disposed thereat, and by being rolled on the skin, the application portion may sequentially press the microneedles corresponding to the protrusions in a direction in which the application portion rotates and sequentially apply the microneedles to the skin.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a perspective view illustrating a microneedle patch including a microneedle according to an embodiment of the present disclosure;

FIG. 2 is a perspective view illustrating a microneedle applicator according to an embodiment of the present disclosure;

FIG. 3 is an exploded perspective view illustrating the microneedle applicator according to an embodiment of the present disclosure;

FIG. 4 is a plan view illustrating an application portion having various curvatures of the microneedle applicator according to an embodiment of the present disclosure;

FIG. 5 is an operational state view of the microneedle applicator according to an embodiment of the present disclosure;

FIGS. 6A to 6I are views illustrating configurations in which various forms of couplers are applied to the microneedle applicator according to an embodiment of the present disclosure;

FIG. 7 is a view illustrating a modification of the microneedle of the microneedle applicator according to an embodiment of the present disclosure;

FIGS. 8A to 8C are views illustrating configurations in which a protrusion is applied to the microneedle applicator according to an embodiment of the present disclosure;

FIG. 9 is a view illustrating a microneedle applicator according to another embodiment of the present disclosure;

FIG. 10 is a view illustrating a feeder of the microneedle applicator according to the other embodiment of the present disclosure;

FIG. 11 is a view illustrating an operation of the microneedle applicator according to the other embodiment of the present disclosure; and

FIGS. 12A and 12B are graphs showing results of measuring infiltration rates and sizes of infiltration holes according to changes in a length and a radius of curvature of a microneedle.

### DETAILED DESCRIPTION OF EXEMPLARY EMBODIMENTS

Hereinafter, exemplary embodiments of the present disclosure will be described in detail with reference to the accompanying drawings in order to enable one of ordinary skill in the art to which the present disclosure pertains to easily embody the disclosure. The present disclosure may be implemented in various different forms and is not limited to embodiments described herein. To clearly describe the present disclosure, parts unrelated to the description have been omitted from the drawings, and like reference numerals are given to like or similar elements throughout.

In the specification, terms such as “include” or “have” should be understood as designating that features, number, steps, operations, elements, parts, or combinations thereof exist and not as precluding the existence of or the possibility of adding one or more other features, numbers, steps, operations, elements, parts, or combinations thereof in advance.

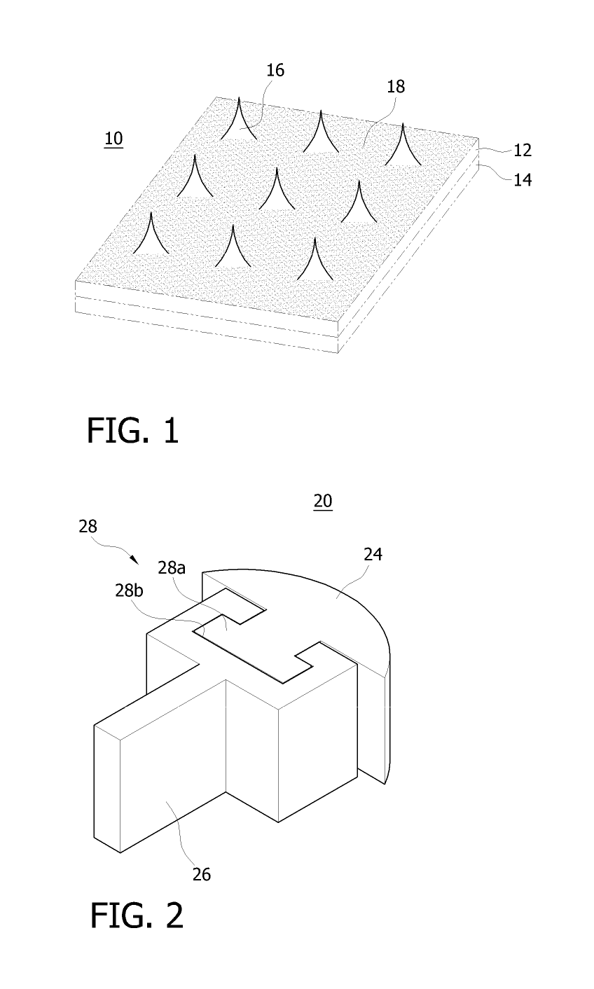

FIG. 1 is a perspective view illustrating a microneedle patch including a microneedle according to an embodiment of the present disclosure.

First, referring to FIG. 1, a microneedle 16 attached to skin and a microneedle patch 10 will be described according to an embodiment of the present disclosure. As illustrated in FIG. 1, the microneedle patch 10 may include an adhesive portion 12 having the microneedle 16 formed at one surface, a supporter 14 disposed at the other surface of the adhesive portion 12, and the microneedle 16.

Here, the supporter 14 may be disposed at the other surface of the adhesive portion 12 at which the microneedle 16 is not formed.

The microneedle patch 10 may be attached to skin by the adhesive portion 12, and when the microneedle 16 is inserted and infiltrated into skin, the supporter 14 may be detached from the microneedle patch 10 and separately collected.

Consequently, an adhesive force between the supporter 14 and the adhesive portion 12 may be weaker than that between the skin and the adhesive portion 12.

The supporter 14 may protect the other surface of the adhesive portion 12 of the microneedle patch 10 which is wound about a roller.

Consequently, the supporter 14 may extend in a longitudinal direction as the microneedle patch 10 and may have a width corresponding to a length of the microneedle patch 10 in a width direction.

Here, the other surface of the adhesive portion 12 may abut the supporter 14, and the microneedle 16 and a support member 15 may be disposed at the one surface of the adhesive portion 12 facing the skin. The adhesive portion 12 may extend in the longitudinal direction of the microneedle patch 10, and a plurality of microneedles 16 may be disposed in an array on the one surface of the adhesive portion 12.

The one surface of the adhesive portion 12 may be adhered to the skin and fix the positions of the microneedles 16 inserted and infiltrated into the skin. For this, an adhesive material for providing an adhesive force upon contact between the microneedles 16 and the skin may be further applied on the one surface of the adhesive portion 12.

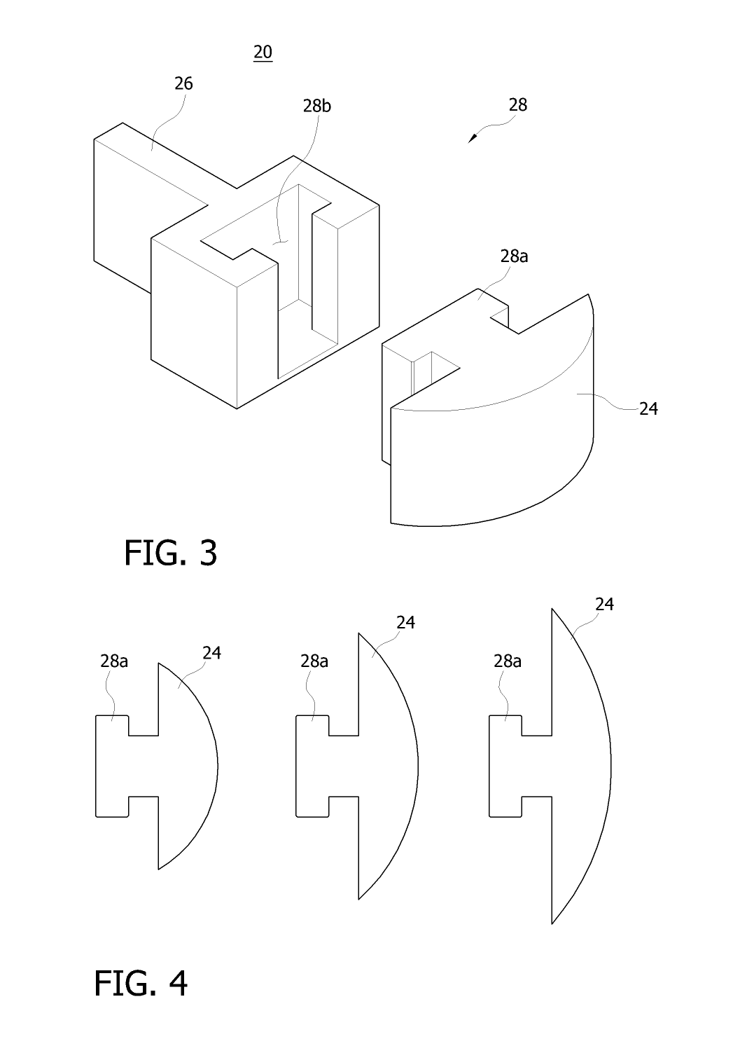

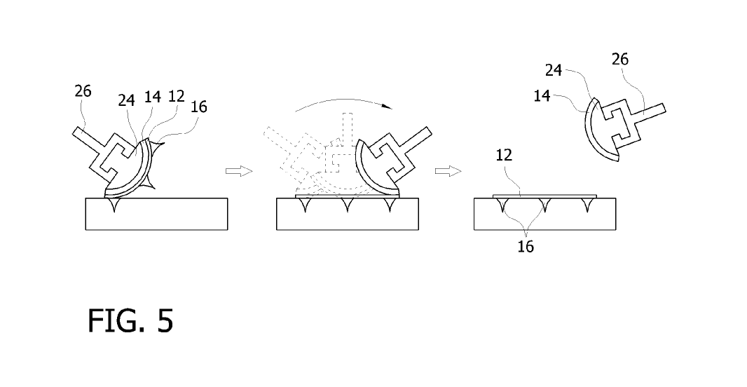

FIG. 2 is a perspective view illustrating a microneedle applicator according to an embodiment of the present disclosure, FIG. 3 is an exploded perspective view illustrating the microneedle applicator according to an embodiment of the present disclosure, FIG. 4 is a plan view illustrating an application portion having various curvatures of the microneedle applicator according to an embodiment of the present disclosure, and FIG. 5 is an operational state view of the microneedle applicator according to an embodiment of the present disclosure.

Hereinafter, a microneedle applicator 20 according to an embodiment of the present disclosure will be described with reference to FIGS. 2 to 5.

The microneedle applicator 20 according to the embodiment of the present disclosure is for inserting and infiltrating the microneedles 16 into skin. Specifically, the microneedle applicator 20 may be used to attach the microneedles 16 themselves, which are not in the form of a patch, to the skin or to attach the microneedle patch 10, which is provided in the form of a patch illustrated in FIG. 1, to the skin.

The microneedle applicator 20 according to the embodiment of the present disclosure may include an application portion 24, a connector 28, a presser 26, and a coupler 22.

Here, the application portion 24 presses the microneedles 16 or one surface of the microneedle patch 10 having the microneedles formed at the other surface thereof for the microneedles 16 to be inserted and infiltrated into skin.

The application portion 24 may extend in the longitudinal direction of the microneedle patch 10 and have a width relatively larger than that of the microneedle patch 10 when the microneedles 16 are provided in the form of a patch. This is to entirely apply the microneedle patch 10 to the skin.

Specifically, according to an embodiment of the present disclosure, an outer surface of the application portion 24 corresponding to and abutting the microneedle patch 10 may be a curved surface having a curvature of a predetermined size. A cross-section of the curved surface may have any one of an arc shape, an elliptical shape, and a parabolic shape when viewed in the longitudinal direction of the application portion 24.

In an embodiment of the present disclosure, because the application portion 24 has the curved surface, the microneedles 16 may be inserted and infiltrated into skin by rolling the curved surface, which is the outer surface of the application portion 24, while the curved surface is partially in contact with the skin.

Here, rolling refers to motion in which rotary motion and parallel advancing motion are combined. The application portion 24 simultaneously rotates so that the curved surface comes into contact with the skin and advances in a parallel direction along a portion on the skin to which the microneedles 16 are to be attached.

Here, the microneedles 16 may be disposed between the curved surface and the portion of the skin to which the microneedles 16 are to be attached.

Consequently, while being disposed corresponding to the skin, the microneedles 16 may be inserted and infiltrated into the skin by the rolling of the application portion 24 included in the microneedle applicator 20.

Here, the microneedles 16 may be directly formed at an outer portion of the curved surface of the application portion 24 included in the microneedle applicator 20.

A type of the microneedle patch 10 in which the plurality of microneedles 16 are formed in an array on the supporter 14 may be disposed at the outer portion of the curved surface of the application portion 24 included in the microneedle applicator 20.

Here, the microneedle applicator 20 according to an embodiment of the present disclosure may further include the coupler 22 formed on the application portion 24 to couple the microneedles 16 or the microneedle patch 10 to the application portion 24.

Here, the coupler 22 may be formed of an adhesive layer. The adhesive layer may have the microneedles 16 formed at one surface or be adhered and coupled to the supporter 14 of the microneedle patch 10. In both of the cases, the microneedles 16 may be disposed to face the skin.

Consequently, the microneedles 16 may be sequentially inserted and infiltrated into the skin by rolling the application portion 24 on the skin.

That is, as illustrated in FIG. 5, the microneedles being sequentially inserted and infiltrated into the skin refers to a case in which, as the application portion 24 disposed corresponding to the skin is rolled, the outer portion on the curved surface of the application portion 24 initially comes into linear contact with the skin in a width direction, the microneedles 16 disposed at the contact portion between the outer portion and the skin are inserted and infiltrated into the skin, and then the outer portion of the application portion 24 coming into linear contact with the skin is moved in a direction in which the application portion 24 advances.

Here, both surfaces of the adhesive layer may have an adhesive force. That is, the adhesive layer may include one surface which has the microneedles 16 formed or is adhered to the supporter 14 of the microneedle patch 10 and an opposite surface adhered to the curved surface of the application portion 24.

Here, the adhesive force of the one surface to which the microneedles 16 are adhered may be weaker than that of the opposite surface of the adhesive layer, and in such a case, the microneedles 16 may be inserted and infiltrated into the skin by rolling the application portion 24, and then the adhesive layer may remain at the application portion 24. That is, only the microneedles 16 may be inserted into the skin without the adhesive layer being attached to the skin.

When the adhesive force of the one surface to which the microneedle patch 10 is adhered is weaker than that of the opposite surface of the adhesive layer, the adhesive layer may remain at the application portion 24, and the microneedle patch 10 may be attached to the skin together with the microneedles 16.

The microneedles 16 may be formed at the outer surface of the application portion via the adhesive layer, or the supporter 14 of the microneedle patch 10 may be adhered or coupled to the application portion 24.

The supporter 14 of the microneedle patch 10 may be formed of the adhesive layer or the coupler 22.

The coupler 22 may detachably couple the application portion 24 to the microneedles 16 or the microneedle patch 10.

However, in an embodiment of the present disclosure, the coupler 22 is not necessarily limited to the adhesive layer, and the coupler 22 may be a configuration capable of physically coupling the application portion 24 to the microneedles 16 or the microneedle patch 10.

Specifically, the application portion 24 may be detachably coupled to the microneedle patch 10 by an embossed structure at which a coupling protrusion or the like is formed.

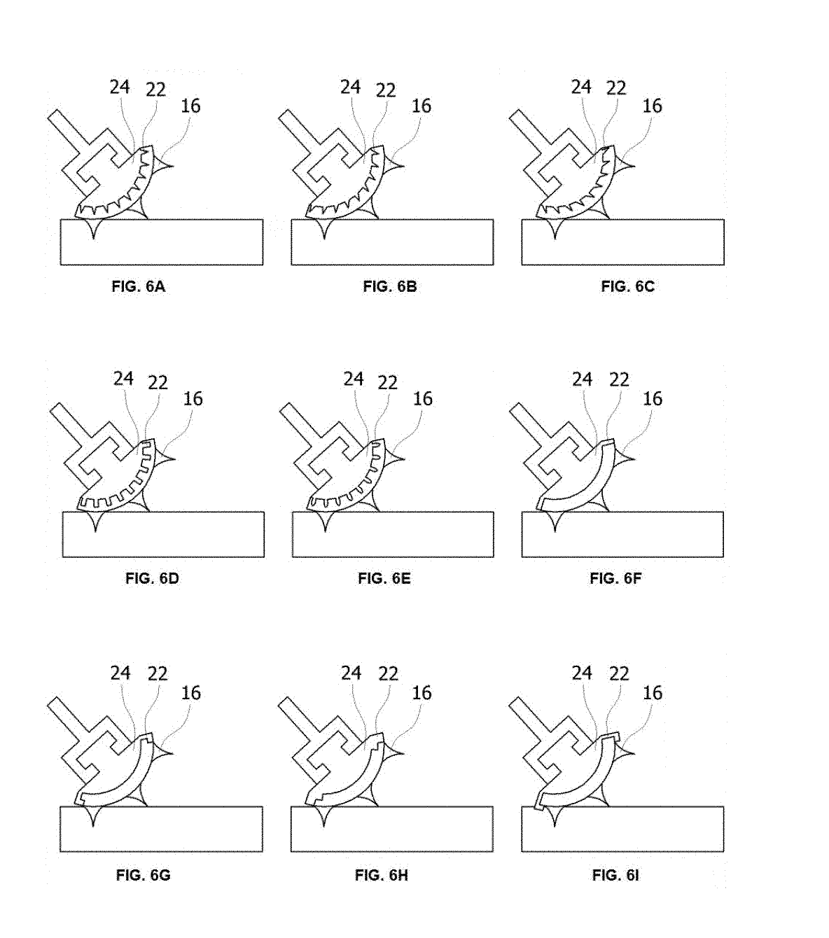

FIGS. 6A to 6I are views illustrating various structures of the coupler of the microneedle applicator according to an embodiment of the present disclosure.

Referring to FIG. 6A, according to an embodiment of the present disclosure, the coupler 22 may include a coupling protrusion protruding from the curved surface of the application portion 24. Here, to correspond to and be coupled to the coupling protrusion, the supporter 14 of the microneedle patch 10 may include a protrusion accommodating groove so that the coupling protrusion is accommodated therein.

The coupling protrusion protruding outward in a radial direction from the curved surface of the application portion 24 may correspond to and be coupled to the protrusion accommodating groove of the supporter 14 of the microneedle patch 10, and the microneedle patch 10 and the application portion 24 may be detachably coupled to each other by the coupling protrusion and the protrusion accommodating groove.

Here, the coupling protrusion may have a triangular cross-section whose outer diameter gradually decreases toward a distal end as illustrated in FIGS. 6A, 6B, and 6C, have a cross-section whose outer diameter is constant as illustrated in FIG. 6D, or have an elliptical cross-section as illustrated in FIG. 6E.

Here, the coupling protrusion may protrude outward in a radial direction as illustrated in FIG. 6A or obliquely protrude toward one direction as illustrated in FIGS. 6B and 6C.

That is, as illustrated in FIG. 6B, the coupling protrusion may extend in an opposite direction of a direction in which the application portion 24 is rolled. Specifically, the direction in which the application portion 24 is rolled refers to a direction in which the application portion 24 is operated to apply the microneedles 16 on the skin. In the case of FIG. 6c, the direction in which the application portion 24 is rolled refers to the clockwise direction.

When the coupling protrusion extends in the counterclockwise direction on the curved surface of the application portion 24, the coupling protrusion of the application portion may be easily detached from the protrusion accommodating groove formed at the microneedle patch when the application portion 24 is rolled clockwise.

Accordingly, the coupling protrusion may be more easily applied when the microneedle patch 10 and the microneedles 16 are attached to the skin together.

Conversely, in a case in which the coupling protrusion obliquely extends in the clockwise direction, which is the same direction as that in which the application portion 24 is rolled, as illustrated in FIG. 6B, the coupling protrusion of the application portion is locked in the protrusion accommodating groove formed in the microneedle patch and generates a locking force when the application portion 24 is rolled clockwise.

Accordingly, when the application portion 24 is rolled clockwise, the coupling protrusion and the protrusion accommodating groove may not be detached from each other. Specifically, the coupling protrusion may remain coupled to the protrusion accommodating groove formed in the supporter 14 of the microneedle patch 10 such that the supporter 14 of the microneedle patch 10 remains at the application portion 24 without change, and only the microneedles 16 are inserted and infiltrated into the skin.

Particularly, when, depending on the type of the microneedles 16, a speed at which the microneedles 16 are dissolved into the body is low and thus the microneedle patch 10 needs to be attached to the skin for a long period, by infiltrating only the microneedles and detaching the microneedle patch from the application portion, an inconvenience in use that may occur when the microneedle patch 10 is attached to the skin for a long period may be mitigated.

As illustrated in FIGS. 6F, 6G, 6H and 6I, the coupler 22 may also be formed of a locking step that abuts both ends of the microneedle patch having the microneedles 16 and generates a locking force.

The locking step of the coupler 22 may perpendicularly protrude from both ends of the application portion respectively corresponding to one end and the other end of the curved surface of the application portion or may be bent or stepped toward the microneedle patch.

The microneedle applicator 20 according to an embodiment of the present disclosure may include the presser 26 configured to provide an external force, which is a pressing force of a predetermined strength, to the application portion 24.

Here, although the presser 26 may be generally operated by an operator, the presser 26 may also be rotated at a predetermined angle in any one direction by an electric or mechanical actuator.

The microneedle applicator 20 according to an embodiment of the present disclosure includes the application portion 24 whose outer surface corresponding to the skin is the curved surface having a predetermined curvature. The application portion needs to have curved surfaces of various forms depending on an area or a length of a portion of the skin to which the microneedles 16 are to be applied.

That is, when an area of the portion to which the microneedles 16 are to be attached is larger than that of the curved surface of the application portion 24, a task in which the application portion 24 is rolled to attach the microneedles 16 may have to be performed several times. Consequently, in an embodiment of the present disclosure, the application portions 24 having various areas, sizes, or curvatures may be replaceably coupled to the presser 26.

Specifically, as illustrated in FIGS. 2 and 3, the microneedle applicator 20 according to an embodiment of the present disclosure includes the connector 28 so that the application portions 24 of various sizes may be easily replaced.

Here, the application portion 24 and the presser 26 should be connected by the connector such that the application portion 24 and the presser 26 are integrally movable in the direction in which the application portion 24 is rolled, and the application portion 24 may be coupled to be detachable from the presser 26 configured to provide the external force.

That is, by being integrally coupled to each other or being integrally connected and coupled to each other via the connector, the application portion 24 and the presser 26 may be rotated at a predetermined angle with respect to the skin in the clockwise direction or the counterclockwise direction shown in the drawings.

Here, the connector 28 may include a connection groove 28b formed at one end of the presser 26 and a connection member 28a formed at one end of the application portion 24. The presser 26 and the application portion 24 may be integrally connected to each other by coupling between the connection groove and the connection member.

The reason for allowing the application portion 24 and the presser 26 to be detachable from each other is to change a size of a curvature of the curved surface of the application portion 24 depending on the type of the microneedles 16 or a length or an area of a portion of the skin to which the microneedles 16 are to be attached.

That is, as illustrated in FIG. 4, because the application portions 24 whose curved surfaces have various lengths and curvatures have the connector 28 of the same form, the application portions 24 may be easily coupled to and detached from the presser 26.

In comparison to the related art, the microneedle applicator 20 according to an embodiment of the present disclosure has the following advantageous effects.

Conventionally, to attach a microneedles to skin, the microneedle or a microneedle patch is pressed in a direction perpendicular to skin to which the microneedle or the microneedle patch are to be attached. However, this requires a strong force, and because the force is dispersed throughout the microneedle patch, efficiency of attaching the microneedle or the microneedle patch is degraded.

Conversely, to more efficiently attach a microneedle or a microneedle patch, the microneedle applicator 20 according to an embodiment of the present disclosure may include an application portion formed of a curved surface. In this way, unlike the conventional case, the microneedle or the microneedle patch may be uniformly pressed in the radial direction. In this way, instead of being dispersed throughout the portion of the skin to which the microneedle or the microneedle patch is to be attached, the pressing force may be concentrated in a direction in which the application portion advances.

Further, conventionally, in a process in which a microneedle or a microneedle patch entirely comes into surface contact with a portion of skin to which the microneedle or the microneedle patch is to be attached and the microneedle or the microneedle patch is infiltrated into the skin, great pain is caused to a patient, and a strong driving force or external force is required for infiltrating the microneedle into the skin.

However, by the microneedle applicator 20 according to an embodiment of the present disclosure, a microneedle partially comes into linear contact with skin, and the microneedle or a microneedle patch may be efficiently inserted and infiltrated into the skin even with a small force.

That is, because the pressing force for pressing the microneedle or the microneedle patch may be concentrated on a portion of the skin to which the microneedle is to be attached, the microneedle may be easily inserted and infiltrated into the skin, and the microneedle patch may be easily attached to the skin.

Although the microneedle applicator 20 according to an embodiment of the present disclosure has been illustrated and described as being formed of an application portion having a semi-spherical cross-section, embodiments are not limited thereto, and the application portion may also be formed in the shape of a cylindrical roller.

Specifically, the application portion 24 may be formed of a cylindrical roller, and as the roller is rolled on a skin surface, the microneedle may be inserted and infiltrated into the skin, and the microneedle patch 10 may be attached to the skin. In such a case, the roller-shaped application portion, which is integrally connected to the presser or connected to the presser via the connector, may be rotated at 360° in one direction.

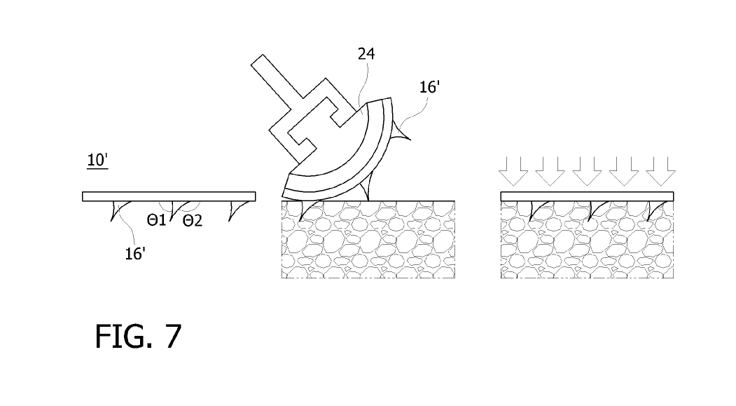

FIG. 7 is a view illustrating a modification of the microneedle of the microneedle applicator according to an embodiment of the present disclosure.

Hereinafter, referring to FIG. 7, a modification of microneedles 16′ or a microneedle patch 10′ easily attached to the application portion 24 of the microneedle applicator 20 according to an embodiment of the present disclosure will be described.

Specifically, the microneedles 16′ may be formed such that two angles θ1 and θ2 formed between the microneedles 16′ and the adhesive portion 12 are different from each other. That is, a distal end of the microneedles may be obliquely formed in one direction.

Here, the microneedles may be formed to have a substantially scalene triangular-shaped cross-section in which a front angle θ1 is formed smaller than a rear angle θ2. In this way, when attaching the microneedles 16′ or the microneedle patch 10′ by the microneedle applicator 20 including the application portion 24 having the curved surface, the microneedles 16′ may be inserted into corresponding skin, which is substantially horizontal to the microneedles 16′, while the microneedles 16′ are almost perpendicular to the skin.

Specifically, the microneedles illustrated in FIG. 7 may extend such that a sharp distal end thereof is obliquely formed in the clockwise direction, which is the same direction as that in which the application portion is rolled.

As described above, because the application portion 24 of the microneedle applicator 20 is formed of the curved surface, each of the microneedles 16′ may be inserted into skin, which is substantially horizontal to the microneedles 16′, in a direction almost perpendicular to the skin when the microneedles are infiltrated into the skin by the rolling of the application portion. In this way, the microneedles may be prevented from being broken, and an infiltration rate of the microneedles may be improved.

Consequently, when the microneedles 16′ are obliquely formed in one direction, the microneedles 16′ may be inserted into skin in a direction perpendicular to the skin surface.

Here, specifically, the two angles θ1 and θ2 may be differently determined according to a curvature of the application portion 24 formed of a curved surface.

Here, a general microneedle (θ1=θ2) as well as the microneedles with changed angles illustrated in FIG. 7 may be attached to skin by the microneedle applicator according to an embodiment of the present disclosure.

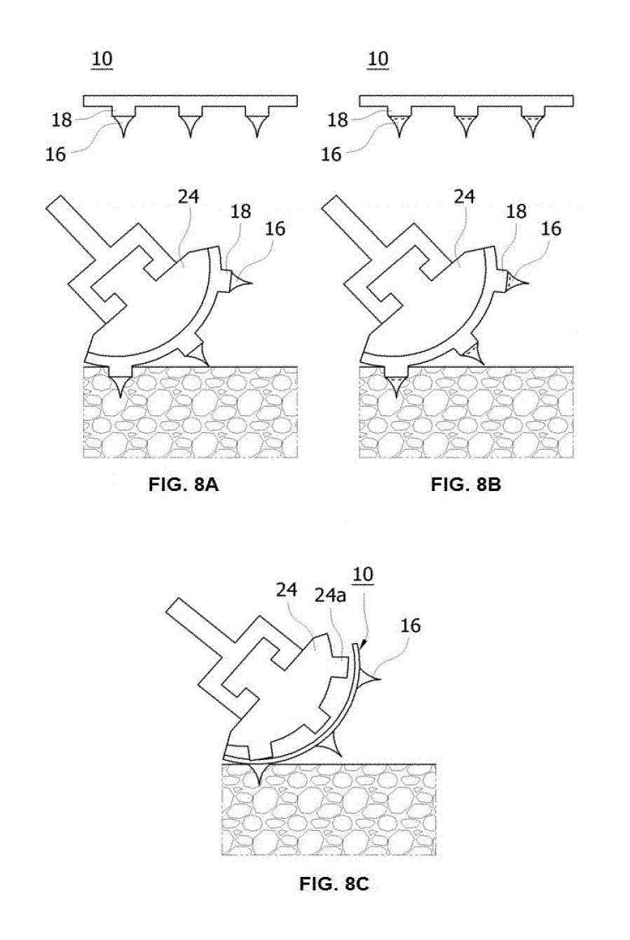

FIGS. 8A, 8B, and 8C are views illustrating configurations in which a protrusion is applied to the microneedle applicator according to an embodiment of the present disclosure.

Referring to FIGS. 8A and 8B, the microneedle patch 10 may include protrusions 18 protruding to a predetermined height from the supporter or the adhesive portion of the microneedle patch 10 so that the microneedles are disposed at positions in one-to-one correspondence with the microneedles.

The protrusions 18 may be formed such that ends thereof at which the microneedles are disposed have a flat cross-section or a semi-spherical cross-section.

Referring to FIG. 8C, the application portion 24 may include protrusions 24a protruding to a predetermined height from the curved surface so that the microneedles are disposed at positions in one-to-one correspondence with the microneedles.

Accordingly, when the microneedles 16 are infiltrated into the skin by the pressing force transmitted to the application portion via the presser, the pressing force may be concentrated on the protrusions 18 and 24 corresponding to the microneedles, thereby improving the infiltration rate of the microneedles, and a depth at which the microneedles 16 are infiltrated into the skin may be adjusted by varying a height to which the protrusions protrude from the microneedle patch or the application portion.

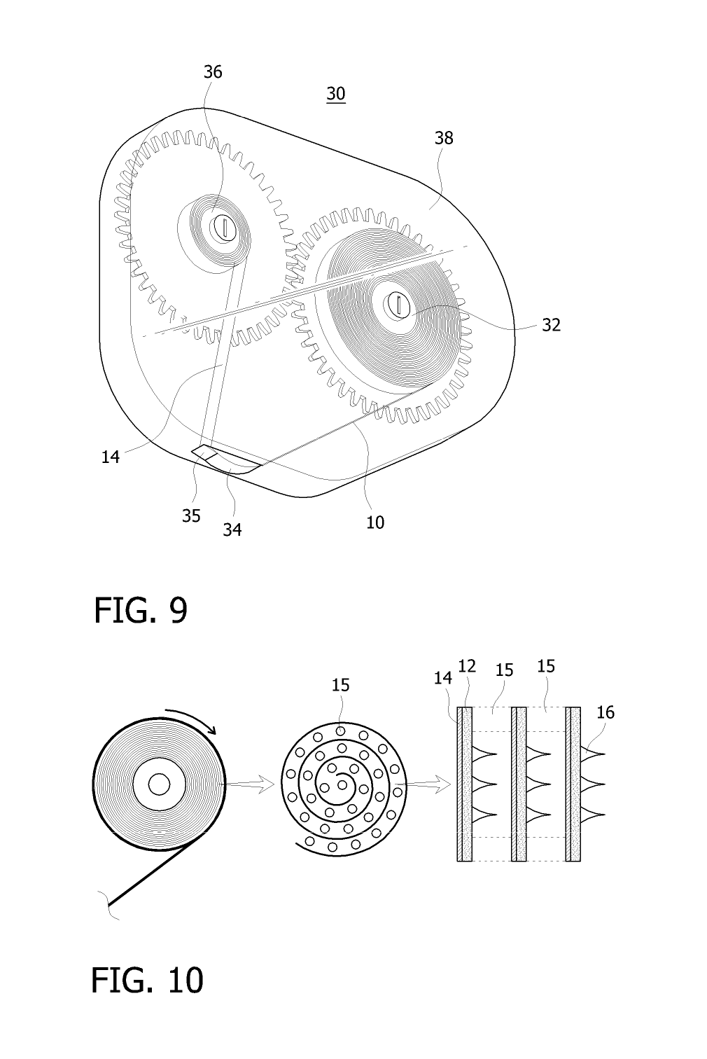

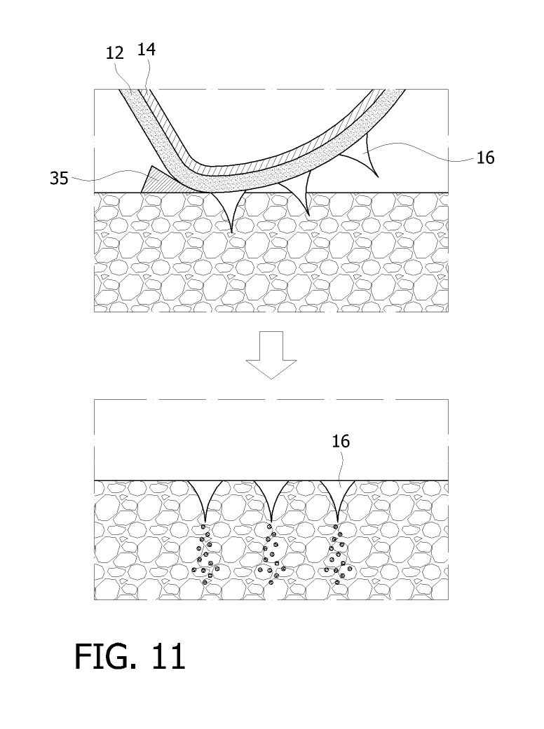

FIG. 9 is a view illustrating a microneedle applicator according to another embodiment of the present disclosure. FIG. 10 is a view illustrating a feeder of the microneedle applicator according to the other embodiment of the present disclosure. FIG. 11 is a view illustrating an operation of the microneedle applicator according to the other embodiment of the present disclosure.

Hereinafter, referring to FIGS. 9 to 11, a microneedle applicator 30 according to the other embodiment of the present disclosure will be described.

The microneedle applicator 30 according to the other embodiment of the present disclosure may include a case 38, a feeder 32, a collector 36, a blade 35, and an application portion 34.

In the other embodiment of the present disclosure, microneedles are provided in the form of a patch and wound in the form of a roll.

Here, as illustrated in FIG. 10, the microneedle patch 10 provided by being wound about a roll may include the support member 15. In this way, even when the microneedle patch 10 is wound in the form o a roll, the microneedle patch 10 and the microneedles 16 adjacent to each other do not come into contact such that the microneedles 16 may be protected.

In the other embodiment of the present disclosure, as illustrated in FIG. 9, the case 38 may have the feeder 32 and the collector 36 disposed therein, and a portion of the application portion 34 may be exposed outward from the case 38.

Referring again to FIG. 9, the application portion 34 exposed to the outside of the case 38 may be formed of a curved surface as described above, and the microneedle patch 10 unwound from the feeder 32 disposed inside the case 38 may move to the outside of the case 38 and move back into the case 38 through the application portion 34. Here, the feeder 32 and the collector 36 may be fixed to an inner surface of the case 38.

The feeder 32 is for loading the microneedle patch 10 and may be formed as a feeding roller. The microneedle patch 10 may be wound about the feeding roller and loaded. Here, the microneedle patch 10 is wound about the feeder 32 such that the supporter 14 faces inward while the microneedle patch 10 faces outward. This is to allow the microneedle patch 10 unwound from the feeder 32 to face the skin to which the microneedles 16 are to be attached.

In this way, the microneedle patch 10 unwound from the feeder 32 may come into contact with the skin, pressed by the application portion 34, which will be described below, and be attached to the skin.

In the other embodiment of the present disclosure, the application portion 34 presses the microneedle patch 10 unwound from the feeder 32 while bringing the microneedle patch 10 into contact with the skin and attaches the microneedle patch to the skin.

Here, the application portion 34 may have a curved surface extending in the longitudinal direction of the microneedle patch 10. The curved surface may have a cross-section formed in various shapes such as a parabolic shape, an arc shape, and an elliptical shape. The curved surface is for effectively attaching the microneedle patch 10 to a curved portion of skin such as wrinkles.

As the microneedle patch 10 is attached to skin by the application portion 34, the microneedles 16 may be inserted into the skin, and drug inside the microneedles 16 infiltrated into the skin or coated on a surface of the microneedles 16 may be supplied into the body.

As the microneedle patch 10 passes through the application portion 34, the adhesive portion 12 and the supporter 14 disposed at one surface of the microneedle patch 10 may be detached from each other. Accordingly, the adhesive portion 12 and the microneedles 16 disposed on the adhesive portion 12 may be attached to the skin, and the detached supporter 14 may be separately collected through the application portion 34.

Preferably, the application portion 34 of the microneedle applicator 30 according to the other embodiment of the present disclosure may further include the blade 35.

Here, the blade 35 may remove the adhesive portion 12 of the microneedle patch 10 passing through the application portion 34. In this way, only the microneedles 16 may be inserted into the skin without the adhesive portion 12 being attached to the skin surface. That is, a conventional problem in that the adhesive portion 12 has to be attached to the skin until drug is injected from the microneedles or the microneedles are dissolved is solved.

In the other embodiment of the present disclosure, the microneedle applicator 30 may include the collector 36 for efficiently collecting the supporter 14 detached from the microneedle patch 10.

Here, the collector 36 may add tension in the longitudinal direction of the microneedle patch 10. By adding tension, the microneedle patch 10 connected through the feeder 32, the application portion 34, and the collector 36 is easily attached to the skin.

Here, as illustrated in FIG. 9, the collector 36 may be formed of a collecting roller. Here, the collecting roller may be connected to the feeding roller by a gear and the like, engaged with the feeding roller, and rotate in an opposite direction of that in which the feeding roller rotates.

Here, a torsion spring may be further coupled to a rotating shaft of the collecting roller. The torsion spring may be formed to preserve an elastic force in an opposite direction of that in which the supporter 14 is wound about the collecting roller. A spring elastic force is used as the tension in the longitudinal direction of the microneedle patch 10.

Because the elastic force preserved by the torsion spring may continuously provide tension to the microneedle patch 10, the microneedle patch 10 may be kept tense in the longitudinal direction.

In the other embodiment of the present disclosure, more preferably, the microneedle applicator 30 may further include a groove capable of adjusting tension of the torsion spring of the collecting roller. The groove may be formed in a side surface of the microneedle applicator 30. Here, by rotating the collecting roller, to which the torsion spring is coupled, in the groove using a flat-bladed screwdriver, the tension provided by the torsion spring may be adjusted. Here, the tension provided by the torsion spring may be differently determined according to mechanical physical property values of the microneedle patch 10.

Referring to FIG. 11, an operation of the microneedle applicator 30 according to the other embodiment of the present disclosure will be described.

To attach the microneedle patch 10 to skin, first, the application portion 34 may be brought into contact with a portion of the skin into which drug is to be injected through the microneedles 16. Here, because the application portion 34 is formed of a curved surface, the microneedle patch 10 may be brought into contact with the skin without being affected by a bent portion of the skin. Here, the collector 36 may add proper tension to the microneedle patch 10 and keep the microneedle patch 10 tense.

The microneedle patch 10 kept tense may come into contact with the skin, and the supporter 14 detachably disposed at the other surface of the microneedle patch 10 may move toward the collector 36.

The blade 35 formed at the application portion 34 may detach the adhesive portion 12 from the microneedles 16 inserted into the skin and allow only the microneedles 16 to be inserted into the skin.

### EXAMPLE 1

Application portions respectively having a radius of curvature of 40 mm, a radius of curvature of 60 mm, and a radius of curvature of 80 mm and a flat plate-shaped application portion were prepared. Each of the application portions includes microneedles having a length of 300 μm.

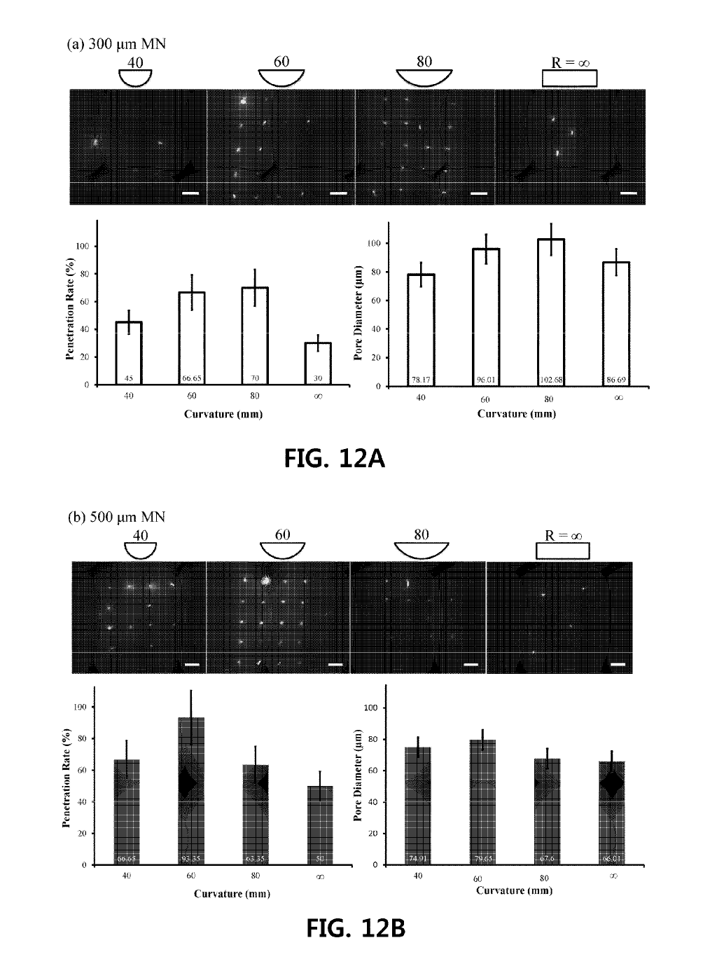

In this state, the microneedles were inserted and infiltrated into skin obtained from a pig, the pig's skin in which fine holes were formed by the microneedles was irradiated with light, and infiltration rates of the microneedles infiltrated into the pig's skin and sizes of infiltration holes were measured. The result thereof is shown in FIG. 12A.

As shown in the graph of FIG. 12A, it can be recognized that, when the microneedles having the length of 300 μm were employed to the application portion having the radius of curvature of 80 mm, a maximum infiltration rate of 70±18.87%, which is relatively higher in comparison to those of the other application portions, and a maximum hole size of 102.68±10.72 μm were obtained.

### EXAMPLE 2

Application portions respectively having a radius of curvature of 40 mm, a radius of curvature of 60 mm, and a radius of curvature of 80 mm and a flat plate-shaped application portion were prepared. Each of the application portions includes microneedles having a length of 500 μm.

In this state, the microneedles were inserted and infiltrated into skin obtained from a pig, the pig's skin in which fine holes were formed by the microneedles was irradiated with light, and infiltration rates of the microneedles infiltrated into the pig's skin and sizes of infiltration holes were measured. The result thereof is shown in FIG. 12B.

As shown in the graph of FIG. 12B, it can be recognized that, when the microneedles having the length of 500 μm were employed to the application portion having the radius of curvature of 60 mm, a maximum infiltration rate of 93.35±18.16% and a maximum hole size of 79.65±6.38 μm were obtained.

Here, the microneedles having the length of 300 μm are infiltrated into skin for skin care, and the microneedles having the length of 500 μm are infiltrated into skin for a medicinal purpose.

Accordingly, the radius of curvature of the application portion included in the microneedle applicator of an embodiment of the present disclosure is preferably set within the range of 60 mm to 80 mm to more efficiently infiltrate the microneedles having the length in the range of 300 μm to 500 μm into skin.

According to an exemplary embodiment of the present disclosure, a microneedle applicator includes an application portion having a curved surface portion so that a microneedle or a microneedle patch can be attached even to a curved portion of skin without pain.

Embodiments of the present disclosure have been described above. However, the spirit of the present disclosure is not limited the embodiments proposed herein. One of ordinary skill in the art who understands the spirit of the present disclosure may easily propose other embodiments within the same scope by adding, changing, and omitting elements, but the embodiments should also be understood as belonging to the scope of the present disclosure.

What is claimed is:

1. A microneedle applicator for infiltrating microneedles into skin and delivering drug into the skin, the microneedle applicator comprising:
an application portion configured to press the microneedles and having, at one side thereof, a curved surface extending in a direction in which the microneedles are applied to the skin;
a coupler having the microneedles disposed toward the skin at one surface thereof and having the other surface thereof at the curved surface; and
a presser extending toward the other side of the application portion and configured to provide a pressing force to the application portion, wherein:
the application portion is rolled on the skin so that the microneedles are sequentially applied to the skin in a longitudinal direction of the curved surface; and
when the microneedles are applied to the skin in the longitudinal direction of the curved surface, the microneedles are detached from the coupler and applied to the skin.

2. The microneedle applicator of claim 1, wherein:
the coupler is formed of an adhesive layer;
the adhesive layer is formed such that an adhesive force at the one surface at which the microneedles are formed is weaker than an adhesive force at the other surface which is disposed at the curved surface; and
when the microneedles are applied to the skin in the longitudinal direction of the curved surface, the microneedles are detached from the adhesive layer.

3. The microneedle applicator of claim 1, further comprising a supporter having one surface at which the microneedles are supported and the other surface detachably coupled to the coupler,
wherein, when the microneedles are applied to the skin in the longitudinal direction of the curved surface, the supporter is detached from the coupler so that the microneedles are applied to the skin.

4. The microneedle applicator of claim 3, wherein:
the coupler includes a coupling protrusion protruding outward from the curved surface in a radial direction; and
a protrusion accommodating groove corresponding to the coupling protrusion is formed at the other surface of the supporter coupled to the coupler for the coupling protrusion to be inserted thereinto and the coupler and the supporter to be coupled.

5. The microneedle applicator of claim 4, wherein:
the coupling protrusion protrudes in the same direction as a direction in which the application portion rolls; and
when the application portion moves toward any one side in the longitudinal direction of the curved surface, the microneedles are applied to the skin, and the supporter remains coupled to the coupler.

6. The microneedle applicator of claim 1, wherein:
the application portion allows the microneedles disposed between the coupler and the skin to come into linear contact with the skin and applies the microneedles to the skin; and
the application portion is rolled on the skin so that a line at which the skin and the microneedles come into contact moves along the curved surface of the application portion.

7. The microneedle applicator of claim 1, further comprising a connector configured to detachably connect the application portion and the presser,
wherein, by being rolled on the skin, the application portion sequentially presses the microneedles along a direction in which the application portion rotates and sequentially applies the microneedles to the skin.

8. The microneedle applicator of claim 1, wherein a longitudinal cross-section of the curved surface has any one of an arc shape, an elliptical shape, and a parabolic shape.

9. The microneedle applicator of claim 1, comprising:
a supporter coupled to one surface of an adhesive portion having the microneedles formed at the other surface; and
protrusions protruding to a predetermined height from the supporter or the adhesive portion at positions in one-to-one correspondence with the microneedles so that the microneedles are disposed thereat,
wherein, by being rolled on the skin, the application portion sequentially presses the microneedles corresponding to the protrusions in a direction in which the application portion rotates and sequentially applies the microneedles to the skin.

10. The microneedle applicator of claim 1, wherein:
the application portion includes protrusions protruding to a predetermined height from the curved surface at positions in one-to-one correspondence with the microneedles so that the microneedles are disposed thereat, and;
by being rolled on the skin, the application portion sequentially presses the microneedles corresponding to the protrusions in a direction in which the application portion rotates and sequentially applies the microneedles to the skin.
